(19) United States
(12) Patent Application Publication
(10) Pub. No.: US 2023/0330298 A1
(43) Pub. Date: Oct. 19, 2023

(54) Aromatherapeutic Honey Bandage

(71) Applicant: Mark Edward Fenzl, Sarasota, FL (US)

(72) Inventor: Mark Edward Fenzl, Sarasota, FL (US)

(21) Appl. No.: 17/868,804

(22) Filed: Jul. 20, 2022

Related U.S. Application Data
(60) Provisional application No. 63/372,968, filed on Apr. 19, 2022.

(51) Int. Cl. A61L 15/40; A61M 21/00; A61L 15/42; A61F 15/00

(52) U.S. Cl. CPC A61L 15/40; A61M 21/00; A61L 15/42; A61F 15/002; A61M 2021/0016

(57) ABSTRACT

The present invention relates to an improved aromatherapeutic, moist, sterile, medium to large wound bandage that is at least equivocal to a manuka honey bandage in antimicrobial effectiveness with improved wound healing and effect on mood.

### CROSS-REFERENCE TO RELATED APPLICATIONS

This application claims the benefit of U.S. Provisional Application 63/372,011, filed on Feb. 3, 2022. The above application is incorporated herein in reference to aromatherapeutic honey bandages.

This application claims the benefit of U.S. Provisional Application 63/372,001, filed on Feb. 4, 2022. The above application is incorporated herein in reference to a moist honey bandage.

This application claims the benefit of U.S. Provisional Application 63/372,968, filed on Apr. 19, 2022. The above application is incorporated herein in reference to using orange blossom honey for a wound bandage and a picture of an orange or orange blossom on the bandage or packaging.

### FIELD

The present disclosure relates to a medical product and, more particularly, a moist bandage that increases the rate of wound healing and through aromatherapy provides a positive mood.

### BACKGROUND

This section provides background information related to the present disclosure which is not necessarily prior art.

The present invention relates to an improved natural bandage, but is moist and provides a positive mood through aromatherapeutic means. There are natural bandages on the market. Many of them contain dry Manuka honey. The problem with Manuka honey bandages is Manuka honey is limited in location to New Zealand and Australia, there is a limit to the production of Manuka Honey, it is expensive, the bandages are small, and dry bandages reduce the rate of wound healing. It has been known for decades that moist wounds heal faster than dry wounds. Using a moist, aromatherapeutic honey bandage that is equivalent to manuka bandages in antimicrobial efficacy improves the rate of wound healing and improves mood of the patient and providers.

Two ways a product is protected from microbial spoilage are water activity level and acidity. Products with low water activity level do not have enough water for bacteria or fungus reproduce and cause damage. Acidity or low pH damage microbes, preventing them from damaging the product. Honey naturally has a low water activity level and acidity.

Honey naturally contains bacterial spores that may germinate when the water activity level increases. Adding sporostatic and/or sporicidal natural products to a natural product bandage decreases risk of infection. Monolaurin and glycerol monostearate are monoglycerides that kill bacterial spores. Some aldehydes demonstrate sporostatic and sporicidal effects on Clostridium botulinum spores. Please see Table 1.

TABLE 1
Sporicidal and sporostatic Minimal Inhibitory Concentration
of aldehydes against Clostridium botulinum spores in mM.

| | Sporostatic | Sporicidal |
|---|---|---|
| | Minimal | Minimal |
| | Inhibitory | Inhibitory |
| Aldehydes | Concentration | Concentration |
| Benzaldehyde | 0.6 | 625 |
| Cinnamaldehyde | 9.8 | >2500 |
| Piperonal | 0.6 | >2500 |
| Vanillin | 0.6 | 125 |

Some essential oils are sporicidal. Please see Table 2.

TABLE 2
Sporicidal activity of essential oils against
Bacillus subtilits spores

| Essential Oil | Log Reduction in Spores |
|---|---|
| Peppermint | 2.6 |
| Bergamot | 2.5 |
| Palmarosa | 2.4 |
| Lemongrass | 2.1 |
| Eucalyptus | 2.0 |

Studies have demonstrated that wounds with moist dressings heal significantly faster than wounds with dry dressings as seen on Table 3.

TABLE 3
Comparison of dry vs moist healing.

| Type Dressing | Day | Percentage Healing |
|---|---|---|
| Dry | 1 | 2 |
| Moist | 1 | 18 |
| Dry | 3 | 34 |
| Moist | 3 | 98 |
| Dry | 5 | 72 |
| Moist | 5 | 100 |

The combination of sporostatic aldehydes, sporicidal essential oils, and sporocidal monoglycerides have a synergistic effect on microbial contaminants. Advantageously, non-limiting examples of use of this product may include a plastic bandage with a gauze center and a protective removable layer, a large abdominal bandage, a series of sheets that may be removed from a canister, a roll of gauze in a resealable bag, or wound packing in a bottle with a lid.

### SUMMARY OF THE INVENTION

In concordance with the instant disclosure, this invention is an improved moderate-to-large size, moist, aromatherapeutic honey wound bandage that may be used as a medical product. The bandage may come in a variety of forms and may contain an aldehyde, limonene, linalyl acetate, linalool, and a monoglyceride as a preservative and aromatherapeutic agent. Alternative products may contain benzaldehyde, eucalyptol, piperonal, citral, geraniol, or glycerol monostearate. This product may be equivalent in antimicrobial and wound healing efficacy to Manuka honey bandage, but may cost less.

One example of several potential medical products that does not limit this patent application, is a moist, sterile medical honey gauze roll with vanillin, limonene, linalyl acetate, linalool, and monolaurin stored in a resealable bag.

Another example that does not limit this patent is a moist, sterile medical honey gauze packing strip with vanillin, limonene, linalyl acetate, linalool, and monolaurin on a gauze strip stored in a plastic container with a cap.

Other variations may include a moist, sterile medical abdominal honey bandage with benzaldehyde, lemongrass essential oil, and glycerol monostearate.

These bandages may contain a honey, artificial honey, molasses, syrup, or nectar for better antimicrobial effect or lower cost. They may contain a synthetic or natural anesthetic to decrease pain. They may contain glycerol, carbomer, aloe vera, and/or another humectant to maintain moisture in the bandage.

### DETAILED DESCRIPTION

Definitions: Moist bandage refers to a bandage containing 4% or more water by weight.

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

The present disclosure relates to a honey bandage that is equivalent to manuka honey bandages on the market but is larger, heals wounds faster through appropriate moisture levels, and improves mood through aromatherapy.

One example of several potential medical products that does not limit this patent application, is sterile orange blossom honey bandage with 1.5% vanillin, 0.4% limonene, 0.2% linalyl acetate, 0.2% linalool, and 0.15% monolaurin. This product may be soaked into a 4.5 inch×4.1 yard gauze roll and stored in a resealable bag.

Another example that does not limit this patent application, is sterile orange blossom honey gauze packing with 0.75% vanillin, 0.2% limonene, 0.1% linalyl acetate, 0.1% linalool, and 0.075% monolaurin. This product may be soaked into a 0.5 inch×5 yard gauze packing and stored in a plastic bottle with a cap.

A variation, that does not limit this patent, is a sterile medical honey abdominal pad with 1.5% benzaldehyde, 0.4% lemongrass essential oil, and 0.15% glycerol monostearate on a 12 inch by 16 inch abdominal pad that can be used in the hospital.

It is desirable for the supplement to have a low water activity level. Honey naturally has a low water activity level containing approximately 17% water. Other additional humectants may include nitric acid, dextrose, fructose, glycerol, glycine, glucose, malic acid, salt, sorbitol, sucrose, and tartaric acid. A skilled artisan may select any suitable humectant, as desired. For example, the supplement can have a water activity level of 0.86.

It is also desirable for a product to have a low pH as this inhibits microbes. Honey typically has a pH between 3.5 and 5.5. Acids lower the pH. Some acids, such as formic acid, acetic acid, oxalic acid, nitric acid, lactic acid, succinic acid, tartaric acid, citric acid, propionic acid, sulfhuric acid, benzoic acid, sorbic acid, sulphamic acid, phosphoric acid, mandelic acid, and adipic acid have synergistic antimicrobial effects with hydrogen peroxide against bacteria. Hydrogen peroxide is naturally found in non-mauka type honeys.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

1. A moist, sterile, sporicidal, aromatherapeutic wound or burn bandage containing non-manuka honey and a fragrance that has a therapeutic effect of improving mood.

2. A bandage in claim #1 that contains vanillin.

3. A bandage in claim #1 that contains limonene.

4. A bandage in claim #1 that contains linalyl acetate.

5. A bandage in claim #1 that contains eucalyptol.

6. A bandage in claim #1 that contains piperonal.

7. A bandage in claim #1 that contains citral.

8. A bandage in claim #1 that contains geraniol.

9. A bandage in claim #1 that contains benzaldehyde.

10. A bandage in claim #1 that contains orange blossom honey.

11. A bandage in claim #1 that has a picture or drawing of at least one orange, orange blossom, or lavender flower on the bandage or packaging.

12. A moist, non-manuka wound honey bandage that is stored or sold in the form of a gauze bandage with a surface area of at least 30 square inches.

13. A bandage in claim #12 that is stored in a roll.

14. A preservative containing vanillin, limonene, linalool, linalyl acetate, and a monoglyceride.

15. A wound honey bandage with the preservative in claim #14.
